(19) United States
(12) Patent Application Publication
(10) Pub. No.: US 2020/0016056 A1
(43) Pub. Date: Jan. 16, 2020

(54) Method of subcutaneous aqueous citric acid injection non-surgical skin tightening excluding orbit

(71) Applicant: Vivi Robyn Stafford, Mission Viejo, CA (US)

(72) Inventor: Vivi Robyn Stafford, Mission Viejo, CA (US)

(21) Appl. No.: 15/862,638

(22) Filed: Jan. 5, 2018

(51) Int. Cl. A61K 8/362; A61Q 19/08; A61K 31/194; A61K 9/00; A61K 9/08

(52) U.S. Cl. CPC A61K 8/362; A61Q 19/08; A61K 9/08; A61K 9/0019; A61K 31/194

(57) ABSTRACT

A method for tightening the papillary dermis, reticular dermis, hypodermis, septa, muscle, ground substance and extracellular matrix through the administering of an aqueous solution of citric acid resulting in the reduction of skin tenting, skin folding, loose skin, flaccid skin, crepe skin, skin sag, skin lag, skin droop, laxity, nose droop and skin overhang such as jowl formation, neck laxity; therefore, tightening jaw line, nasal bridge, draping over the zygomatic arch, the neck and other structures of the face and body (excluding structures of the orbit).

### BACKGROUND OF THE INVENTION

This invention was not made with U.S. government support.

Aging consists of a growth phase and a deterioration phase. Deterioration related to aging commences upon the completion of puberty. Deterioration secondary to aging becomes more pronounced in the mid-twenties and by the mid-thirties the aging process often results in irreversible signs in individuals. The irreversible signs of aging often can cause individuals to worry about their appearance and cause stress due to the prospect of appearing older. As a result, individuals purchase over the counter antiaging products, visit aestheticians and seek out advanced and specialized medical care from plastic surgeons, dermatologists, and general practitioners and other skin care and anti-aging specialists and surgeons.

After the age of 40, many individuals show signs of aging they would like to reverse in addition to preventing the progression of which results in a motivating factor for self-improvement. Treatments for aging historically have been pursued by older populations. Non-surgical options however have demonstrated to be appealing to younger individuals that seek not only treatment measures but preventive measures. The signs of aging skin is often resistant to over the counter creams for the reduction of skin tenting, overhang, laxity and other signs of aging.

The surgical alternative for skin tightening is limited to rhytidectomy and variations thereof where incisions are made and skin is excised in appropriate candidates. Additionally, surgical treatments have benefits and risks notwithstanding the financial investment while general anesthesia presents risks for unexpected outcomes. All surgeries are associated with known risks of anesthesia, infection, bleeding, bruising, scarring, as well as the possibility of poor outcome with anticipation of possible discomfort and “down-time” for the patient.

Laser tightening devices that are approved for tightening exist but carry the risk of 1st, 2nd and 3rd degree burns resulting in delayed healing. Furthermore, fat atrophy is also a risk factor in some devices. Presently, there are no skin tightening injections for dermis, reticular dermis, hypodermis, subcutaneous septa that reverse skin tenting, laxity, overhang, sag or signs of aging (excluding the structures proximal to the orbit). Other injection materials that are used in cosmetic treatments not for skin tightening have had side effects such as creating nodules in the hypodermis, causing blindness and skin necrosis. Citric acid in aqueous solution does not have the components for such side effects as the treatment is in an aqueous solution. There is a need in the market, therefore, for a non-surgical and non-laser alternative to address skin tightening with zero risk of skin necrosis or claudication of arteries causing irreversible damage. A citrate solution satisfies this need in that a citrate solution could be used to successfully treat skin conditions of the aging skin without the risk of causing irreversible harm such as arterial claudication. The demand for non-surgical skin tightening has shown to be statistically significant in the cosmetic market.

In spite of procedural risks, the growing appeal of cosmetic medical treatment of said procedures is a testament to the psychological importance of body image and the beneficial outcomes of these procedures as perceived by the patients who seek them. Thus, the significance of a product that corrects signs of aging through skin tightening with little to no risk is vitally important.

### SUMMARY OF THE INVENTION

The present invention is directed to a method for the non-surgical reduction of localized loose skin that is associated with normal aging due to the loss of the integrity of the dermis and hypodermis that supports the epidermis. This treatment excludes treatment in the areas of the orbit. Skin tenting and overhang and laxity can result in an aged appearance. An aqueous solution of citric acid is a treatment for skin tenting, lid laxity, loose skin, sagging skin, overhang and drooping skin with an aqueous solution of citric acid with a viscosity consistent with water at 20 to 25° C. that tightens the neck, and jawline, and other anatomical sites outside of the orbit and structures proximal to the orbit and performs generalized skin tightening in the face and body.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1(a) shows the neck area of a patient before treatment, according to an embodiment of the present invention;

FIG. 1(b) shows the neck area of a patient after treatment, according to an embodiment of the present invention;

FIG. 2(a) shows the neck area of a patient before treatment, according to an embodiment of the present invention;

FIG. 2(b) shows the neck area of a patient after treatment, according to an embodiment of the present invention;

FIG. 3(a) shows the nasolabial area of a patient before treatment, according to an embodiment of the present invention;

FIG. 3(b) shows the nasolabial area of a patient after treatment, according to an embodiment of the present invention;

FIG. 4(a) shows the nasolabial fold area of a patient before treatment, according to an embodiment of the present invention;

FIG. 4(b) shows the nasolabial fold area of a patient after treatment, according to an embodiment of the present invention;

FIG. 5(a) shows the jowl area of a patient before treatment, according to an embodiment of the present invention; and

FIG. 5(b) shows the jowl area of a patient after treatment, according to an embodiment of the present invention.

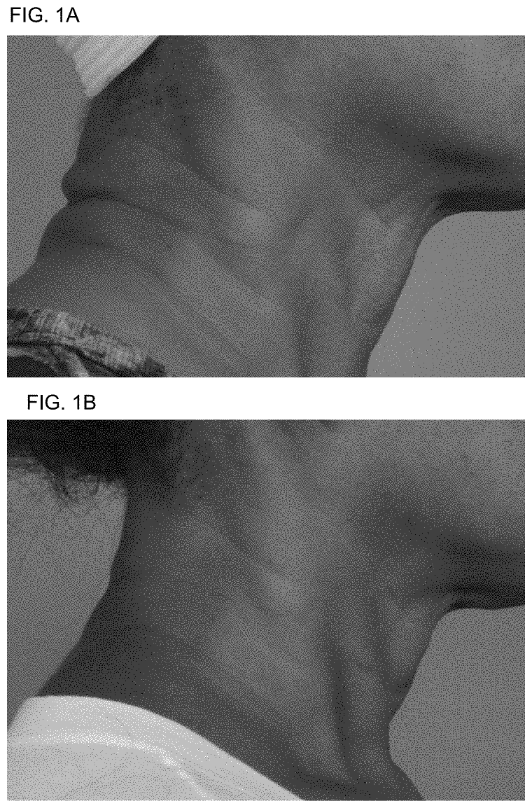

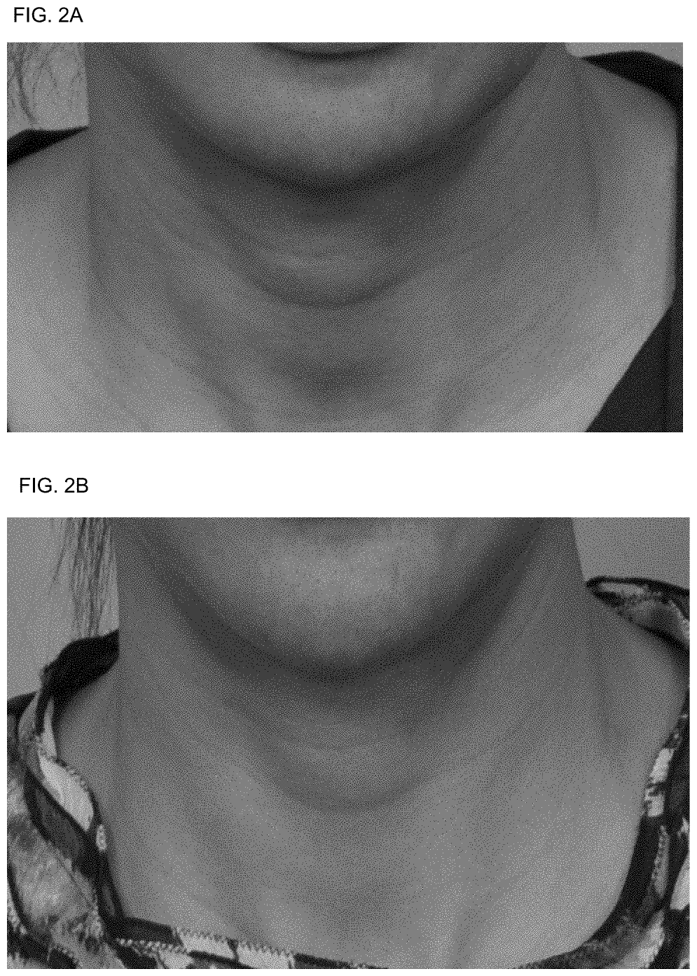

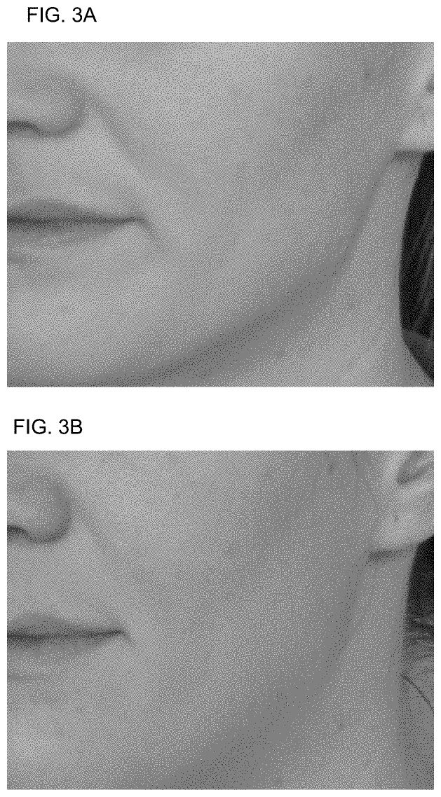

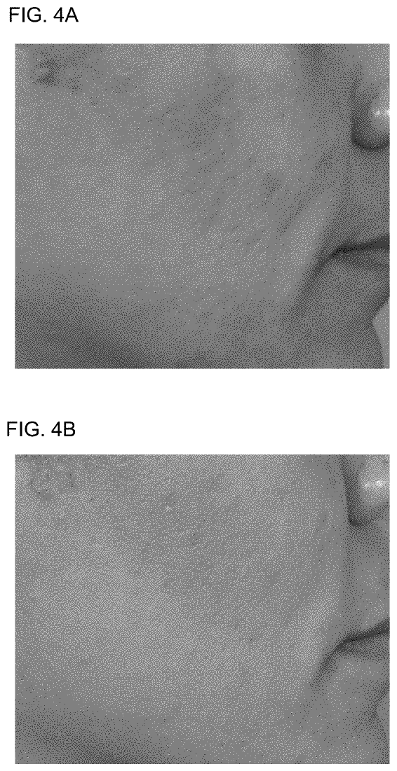

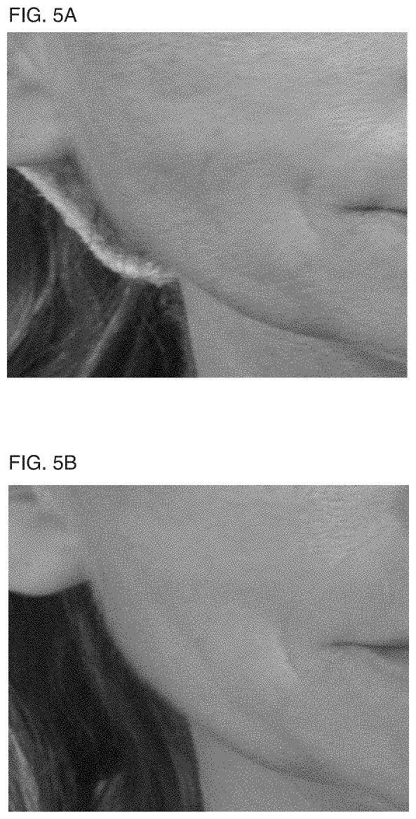

### DETAILED DESCRIPTION OF THE INVENTION

The Invention provides a method for improvement of the signs of the deteriorating aging process of human skin of the face and body comprising administering citric acid or a salt thereof to a patient in need thereof, wherein the administration is via a subcutaneous injection (excluding the orbit) in aqueous solution. The method tightens subcutaneous fat via strengthening of wall proteins located in the hypodermis of the skin. The injection is, for example, in the form of one or more large blebs or small boluses. The method may also include an additional step of measuring one or more improvements in the appearance of the skin with a measuring device. When the citric acid in aqueous solution or salt thereof is placed in the superficial or deep plane of subdermal fat it causes skin tightening by connective tissue contractions, increase support of the subcutaneous tissue, and tensile strength to protein strands below the dermis. The citric acid or salt thereof may be either natural or synthetic.

The compositions, treatment schedules and methods of using said compositions discovered by the inventor effectively reverses the effect of tenting or impending tenting of the skin reverses skin folds and folding and improves the appearance of photo damage and skin crepe. Generally speaking, tenting and impending tenting and tented skin are a well-known beauty problem and the problem can progress to irreversibility if treatment is delayed. Gently pinching the skin gives a baseline of the degree of tenting involved in an individual. The effects of tightening tented and hanging skin with a subcutaneous injection procedure repositions fat and re-compartmentalizes fatty tissue in its more anatomic position. This description does not suggest such effects involving the orbit or proximal to the orbit.

Citrate constituted with isotonic water and dextrose can stimulate elastin tensile strength below the dermis and above and below the muscle in the pre periosteal space to tighten skin, decrease the appearance of aging, and contour deficiencies, fine lines and wrinkles. Citrate is found in the serum of individuals, but also can be manufactured and processed.

The composition of the instant application is based on 100 volume percent calculations. In particular, the composition of the instant application is comprised of: 10 volume percent: 3.2% sodium citrate buffer. In the preferred embodiment, approximately 0.109 ml of sodium citrate buffer is used. Citric acid, having the formula, may also be used: C6H8O7(anhydrous) or C6H8O7 or CH2COOH—C(OH)COOH—CH2COOH

90 volume percent: 36.6% isotonic water, 45% sodium chloride and 6.4% glycerol with trace electrolytes.

In an embodiment of the present invention, sodium citrate powder, or a solution thereof, may be pre-loaded into a 20 cc intravenous vial for dispensing, and combined in an 8 to 10 cc syringe filled with a liquid volume comprising isotonic water, sodium chloride, and glycerol. The injector mixes the ingredients back and forth between two 10 cc syringes until the formula is reconstituted to a dose of 10 ml prior to administration.

The mixture should have a neutral pH prior to injection.

The composition of the instant application for the method of non-surgical skin tightening of the skin with aqueous citric acid injections excluding structures of the orbit is provided for the subcutaneous reversal of skin tenting, sagging, lag, laxity, skin crepe and droop that should be administered only by competent plastic surgeons, dermatologists or other medical doctors and their potentially eligible licensed staff such as state licensed nurses and/or physician assistants. This treatment is of such a benign nature that clearance for injections by licensed vocational nurses may be deemed appropriate thus widening the therapeutic access for those in need of or who desire dermal corrections. Injections into the sub-dermal tissue in patients having skin tenting, sagging or lag of the dermis will tighten and thicken the subdermal fat bands and the hypodermis thereby reducing the appearance of fine lines, wrinkles, and pores.

What is claimed is:

1. A method for improvement of the effects of skin aging through skin tightening comprising via administering citric acid or a salt thereof to a patient in need thereof, wherein said administering is subcutaneous injection including the papillary dermis, reticular dermis, hypodermis, skin fibrous septa, dermal supported muscles, ground substance and extracellular matrix through administering injections of aqueous solutions of citric acid excluding structures of the orbit.

2. The method of claim 1, wherein said administering tightens subcutaneous fat via strengthening wall proteins located in the hypodermis of the dermis to thereby enhancing the appearance of aging skin excluding structures comprising the orbit.

3. The method of claim 2, wherein said method is used fro cosmetic purposes including structures such as perioral, jowls, zygomatic arch, chin, nose, nasolabial folds, jawline regions and not excluding other anatomical sites such as the periumbilical region or the epigastric region.

4. The method of claim 2, wherein said method is used for medically necessary corrective purposes.

5. The method of claim 1, wherein said injection is in the form of one or more blebs or boluses of aqueous solution.

6. The method according to claim 5, wherein said patient suffers from a conditions(s) of aging selected from the group consisting of skin tenting, impending skin tenting, skin laxity, skin sag, skin overhang, effects of oxidative stress, gravitational pull, photo aging, photo damage, crepe skin, loose skin, skin folds, flaccid skin, smoking damage and other effects of extrinsic and intrinsic aging.

7. The method of claim 1, further comprising measuring one or more improvements in the appearance of the skin with a measuring device.

8. The method of claim 1, wherein said citric acid or salt thereof is placed in the superficial plane or deep plains of subdermal fat, papillary dermis, reticular dermis, hypodermis, muscle, matrix, subcutaneous septa thereby causing skin tightening by connective tissue contractions, stimulation of fibroblasts, improved elastic connective tissue, increased support of subcutaneous tissue and providing tensile strength to protein strands below the dermis.

9. The method of claim 1, wherein citric acid or salt thereof is obtained from natural sources is synthetic.

10. The method according to claim 1, wherein said method is administered to a patient in need thereof either before or after the patient undergoes surgery, cosmetic treatments or reconstructive procedures.

11. The method according to claim 1, wherein said method results in the repositioning of fat and/or re-compartmentalizing of fatty tissue, sub-dermally undergoing said method.

12. The method according to claim 1, wherein said method results in the re-positioning of soft tissue relating to the dermis, hypodermis, muscle and extracellular matrix.

13. The method of claim 1, wherein said method has therapeutic applications for skin laxity post bariatric surgery.

14. The method of claim 1, wherein said method has therapeutic applications for reconstructive surgery.

15. The method according to claim 1, wherein said method results in re-positioning of soft tissue relating to the muscles that provide support to the dermis.

16. The method according to claim 1, wherein said method results in fibroblast and elastin up regulation in maintaining dermal structural integrity hence decreasing the effects of aging and other trauma to the visible skin.

17. The method of according to claim 1, wherein said method results in enhancement of subdermal ground substance and reverses oxidative stress and functions as an antioxidant.

18. The method of claim 1, wherein said method results in skin tightening hence diminishing the appearance of pores.

19. The method of claim 1, wherein said method results in skin tightening hence diminishing the appearance of stretch marks and scars.

20. The method of claim 1, wherein said method results in skin tightening, dermal thickening and hypodermal rejuvenation hence improving texture from photo damage, appearance of fine lines and wrinkles and folds.
